(19) United States
(12) Patent Application Publication
(10) Pub. No.: US 2026/0306245 A1
(43) Pub. Date: Oct. 8, 2026

(54) SOFT TISSUE MESH ENVELOPE DEVICE

(71) Applicant: Katherine Ann Weimer, Evergreen, CO (US)

(72) Inventor: Katherine Ann Weimer, Evergreen, CO (US)

(21) Appl. No.: 19/636,584

(22) Filed: Apr. 1, 2026

Related U.S. Application Data
(60) Provisional application No. 63/782,889, filed on Apr. 3, 2025.

(51) Int. Cl. A61F 2/12

(52) U.S. Cl. CPC A61F 2/12; A61F 2210/0004; A61F 2240/001; A61F 2250/0003

(57) ABSTRACT

A biocompatible implantable mesh envelope with a soft tissue filling for use as a soft tissue graft and method of use. The mesh envelope is designed to keep a soft tissue filler material within a desired bounding volume without dispersal to unwanted locations. The mesh envelope has an micro lattice structure that allows for nutrient diffusion across a mesh wall membrane and into inserted cellularized or non-cellularized soft tissue filler. The mesh envelops provides structural support for the filler unlike existing fat grafts or non-cellularized soft tissue fillers and may be sequentially filled to customize a shape to fit an empty tissue cavity.

### CROSS-REFERENCE TO RELATED APPLICATION

The present application claims priority to, and the benefit of, U.S. Provisional Application No. 63/782,889, which was filed on Apr. 3, 2025, and is incorporated herein by reference in its entirety.

### FIELD OF THE INVENTION

The present invention generally relates to an implantable device for augmenting, repairing or replacing soft tissue. More specifically, the present invention relates to a three dimensional mesh envelope that is customizable to fill a defect in soft tissue without allowing the filling material to leach out. Accordingly, the present specification makes specific reference thereto. However, it is to be appreciated that aspects of the present invention are also equally amenable to other like applications, devices and methods of manufacture.

### BACKGROUND

Human soft tissue is widespread throughout the body, and includes muscle, tendons, ligaments, fat, fibrous tissue, lymph and blood vessels, fasciae, and synovial membrane. Human soft tissue is generally very flexible and has the ability to deform and return to its original shape. The mechanical properties of soft tissue and vary significantly from one person to another and on the direction of an applied force. Loss or damage to soft tissue can result from injuries, surgical procedures, and disease. Further, removal of tumors and congenital defects can also result in soft tissue defects.

There are many techniques and procedures to correct soft tissue damage. Tissue engineering uses a combination of cells, engineering, materials methods, and suitable biochemical and physicochemical factors to restore, maintain, improve, or replace different types of biological tissues. A tissue flap using tissue from another part of the body can be used to repair defects. Injectable fillers and hydrogels can be used to restore lost tissue volume. Tissue or polymer scaffolds can be used to provide a framework for new tissue growth. Stem cells can differentiate into different cell types for soft tissue regeneration. Three dimensional (3D) printing techniques can be used to create customized implants.

Unfortunately, there are many challenges and problems with known soft tissue replacement options. Harvesting tissue from donor sites can cause complications and pain. Traditional fat grafts often have complications and generally have low graft survival rates. Injected fat grafts are not able to reconstruct or recreate specific geometric shapes; rather they fill a void and take the shape of the lowest resistance space. Existing techniques struggle to allow for customizable tissue replacement strategies that can be tailored to a specific patient. Many current options require large incisions or invasive implantation techniques.

Accordingly, there is a great need for a way to replace or repair missing or damaged soft tissue. There is also a great need for a patient specific soft tissue replacement implant. There is also a great need for a soft tissue replacement implant that allows for structural support of cellular soft tissue. There is also a great need for a way to keep implanted soft tissue in a desired bounding volume. There is also a great need for a soft tissue implant with a plurality of compartments for that can be filled independently. Finally, there is also a great need for a minimally invasive soft tissue implant.

In this manner, the improved three dimensional mesh envelope of the present invention accomplishes all of the foregoing objectives, thereby providing an easy solution for replacing or repairing missing or damaged soft tissue. A primary feature of the present invention is a patient specific soft tissue replacement implant that allows for structural support of cellular soft tissue. Another feature of the present invention is a soft tissue implant configured to have multiple internal compartments. Another feature of the present invention is a soft tissue implant configured to keep implanted soft tissue in a desired bounding volume. Another feature of the present invention is a soft tissue implant that can be customized in volume and shape in real time during a surgery. Finally, the improved three dimensional mesh envelope of the present invention is capable of being implanted in a minimally invasive surgical procedure.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one embodiment thereof, comprises a three dimensional mesh envelope (3DME) device. The 3DME device may be a plurality of multi-layered compartments, configured as an implantable mesh envelope with an expanding bounding volume that is fillable with soft tissue or soft tissue-like material to the size of the removed tissue or desired tissue replacement during surgery. The 3DME device comprises a three dimensional mesh shaped as an envelope, balloon, disc, or any other three dimensional (3D) bounding volume. The 3DME device comprises at least one compartment. The compartment is constructed from a micro lattice mesh. The 3DME device may further comprise a plurality of additional compartments. Each compartment comprises a port. Each port may comprise a tubing that may be externally accessible either transcutaneously or percutaneously when the 3DME device is implanted. Additionally, each compartment may be externally accessible percutaneously through direct injection/addition of the filler material into the compartment (e.g. without a port). Each compartment is configured to retain a cellularized infill material injected via the respective port. The 3DME device may further comprise a load bearing strut structure within each compartment.

The subject matter in an additional embodiment is a method of repairing a soft tissue defect using a 3DME device. Using a minimally invasive surgical procedure, a collapsed 3DME device is inserted into a soft tissue void via a catheter or surgical instrument. Alternatively, a prefilled 3DME device may be inserted into a soft tissue void. Once positioned, cellularized infill material is injected to each compartment sequentially via the externally positioned ports. Material is injected until a desired size and shape of the 3DME device is achieved.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a perspective view of one embodiment of an exemplary illustration of a three dimensional mesh envelope device of the present invention in accordance with the disclosed architecture.

FIG. 2 illustrates a perspective view of the three dimensional mesh envelope device of the present invention being injected with a filling in accordance with the disclosed architecture.

FIG. 3 illustrates a perspective view of the three dimensional mesh envelope device of the present invention being injected with a filling in accordance with the disclosed architecture.

FIG. 4 illustrates a perspective view of an expanding volume of the three dimensional mesh envelope device of the present invention in accordance with the disclosed architecture.

FIG. 5 illustrates a perspective view of an internal strut structure of the three dimensional mesh envelope device of the present invention in accordance with the disclosed architecture.

FIG. 6 illustrates a side view illustrating nutrient diffusion across a mesh wall membrane of the three dimensional mesh envelope device of the present invention in accordance with the disclosed architecture.

FIG. 7 illustrates an exploded view of the three dimensional mesh envelope device of the present invention being injected through a funnel in accordance with the disclosed architecture.

FIG. 8 illustrates an exploded view of the three dimensional mesh envelope device of the present invention being injected through a funnel in accordance with the disclosed architecture.

FIG. 9 illustrates a side view of the three dimensional mesh envelope device of the present invention accordance with the disclosed architecture.

FIG. 10 illustrates a perspective view of a plurality of compartments of the three dimensional mesh envelope device of the present invention in accordance with the disclosed architecture.

FIG. 11 illustrates a perspective view of a plurality of compartments of the three dimensional mesh envelope device of the present invention in accordance with the disclosed architecture.

FIG. 12A illustrates a side view of a human breast with a tumor and a proposed margin for receiving the three dimensional mesh envelope device of the present invention in accordance with the disclosed architecture.

FIG. 12B illustrates a side view of the human breast with the tumor removed and the proposed margin for receiving the three dimensional mesh envelope device of the present invention in accordance with the disclosed architecture.

FIG. 12C illustrates a side view of the human breast with the three dimensional mesh envelope device of the present invention implanted in accordance with the disclosed architecture.

FIG. 12D illustrates a side view of the human breast with the three dimensional mesh envelope device of the present invention implanted and being filled with the filling in accordance with the disclosed architecture.

FIG. 12E illustrates a side view of the human breast with the three dimensional mesh envelope device of the present invention implanted and expanding to fill a tissue cavity within the excised tissue in accordance with the disclosed architecture.

FIG. 12F illustrates a side view of the human breast with the three dimensional mesh envelope device of the present invention implanted and expanded to completely fill the tissue cavity within the excised tissue in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They do not intend as an exhaustive description of the invention or do not limit the scope of the invention. Additionally, an illustrated embodiment need not have all the aspects or advantages shown. Thus, in other embodiments, any of the features described herein from different embodiments may be combined.

The present invention, in one exemplary embodiment, is a three dimensional mesh envelope (3DME) device. Existing three dimensional (3D) implantable products for soft tissue repair have many deficiencies. There are currently no implantable products that keep a soft tissue graft only in the desired bounding volume location that also allows for nutrient transport through the mesh to enable engraftment of the lipoaspirate. Current products also do not allow for a real-time custom shape to be created immediately in the operating room. There is currently no product designed to prevent a cellularized or non-cellularize soft tissue filler material from dispersing to undesired locations in the body.

Existing open lattice concepts do not contain the inserted cellularized material (i.e. lipoaspirate) injection. Another problem is the need to allow for nutrient diffusion (oxygen, glucose, amino acids, fatty acids and lipids, hormones and growth factors, ions and electrolyte, etc.). Silicone implants do not allow for this. There are no soft tissue implants that allows for structural support of the soft cellular tissue (e.g. lipoaspirate) when needed. A fat graft alone, without a three dimensional mesh envelope (3DME) scaffold does not allow for this. Finally, there is currently no implant that allows for the sequential filling of soft tissue, or soft tissue-like (e.g. lipoaspirate) material into specific regions of the mesh envelope through multiple compartments, or layers, within the mesh envelope.

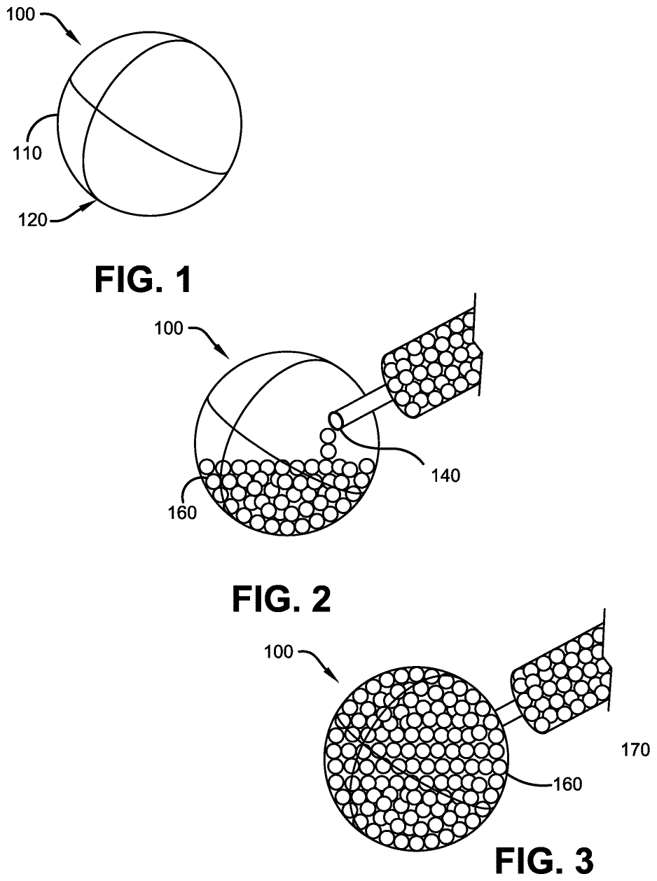

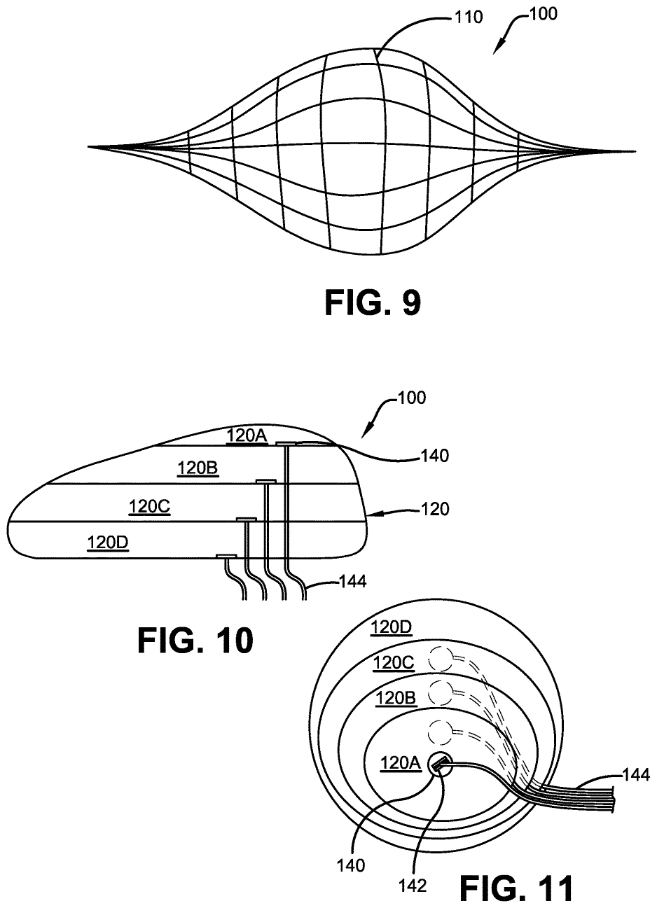

Referring initially to the drawings, FIGS. 1-4 illustrate a three dimensional mesh envelope (3DME) device 100. The 3DME device 100 comprises a mesh wall membrane 110, at least one compartment 120, a filler material 160, and a port 140. The mesh wall membrane 110 may be a micro lattice structure, mesh, or scaffold that is customizable in shape as illustrated in FIG. 9. The mesh wall membrane 110 may be a three dimensionally bio-printed mesh and is a biocompatible soft tissue membrane in one embodiment. Alternatively, the mesh wall membrane 110 may a synthetic or a hybrid membrane. The mesh wall membrane 110 may be absorbable or nonabsorbable depending on the application. The mesh wall membrane 110 is configured to permit nutrient diffusion across the mesh wall membrane 110 while preventing the leaching of the filler material as illustrated in FIG. 6.

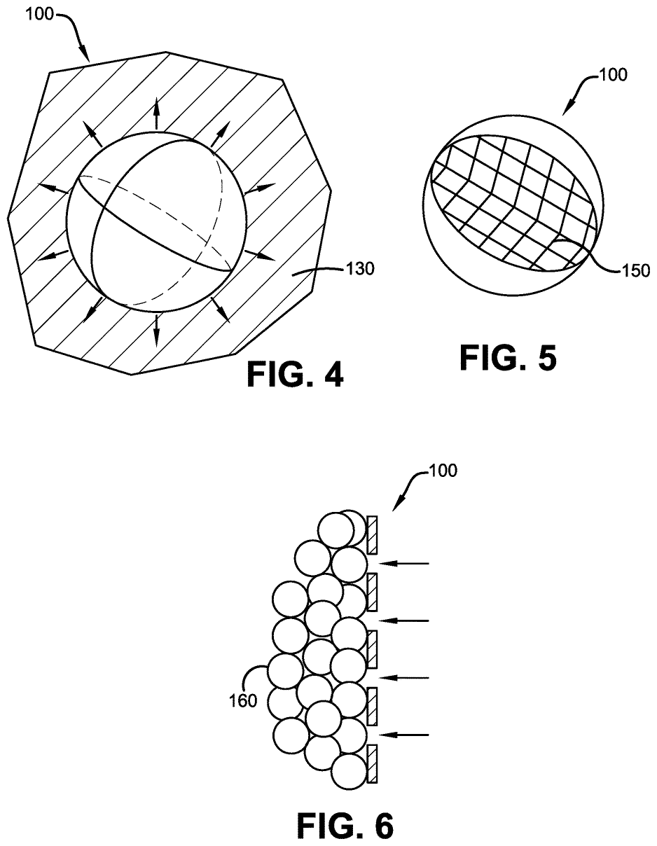

The at least one compartment 120 is formed out of and within the mesh wall membrane 110 and comprises an expandable volume 130 as illustrated in FIG. 4. The at least one compartment 120 is configured to retain the filler material 160 within the expandable volume 130 as illustrated in FIGS. 2 and 3. The filler material 160 may be a harvested adipose or lipoaspirate material or similar soft tissue cellularized or non-cellularized tissue as further described infra.

The port 140 is configured to access the expandable volume 130 within the at least one compartment 120. A syringe 170 or similar filling device may be used to access the port 140 to inject the filler material 160 into the at least one compartment 120.

As illustrated in FIG. 5, the 3DME device 100 may further comprise a plurality of struts 150. The plurality of struts 150 or lattice constructs are positioned within the expandable volume 130 of the at least one compartment 120. The plurality of internal struts 150 may be load bearing struts.

As illustrated in FIGS. 10-11, the 3DME device 100 may alternatively comprise a plurality of compartments 120A-D instead of just at least one compartment 120. Each of the plurality of compartments 120A-D are formed within the mesh wall membrane 110 and each compartment 120A-D comprises an expandable volume 130. Further, each compartment 120A-D comprises a port 140 which may comprise a valve 142 and a tubing 144 for injecting the filler material 160 into the respective compartment 120A-D. The port 140 may also be constructed within the mesh wall membrane 110 and function similarly to a surgically created dialysis shunt, such as arteriovenous (AV) fistula/graft. In an embodiment without a non-organic port, the 3DME device 100 would comprise a small opening functioning as the port 140 that a cannula or needle is inserted into. Then the filler is injected, then the hole is sewn, stapled, etc. closed. The expandable volume 130 of each of each of the plurality of compartments 120A-D is adjustable and may be different. The plurality of compartments 120A-D may be oriented in a layered configuration or may be completely customizable to fit a specific cavity.

The 3DME device 100 is essentially a single or multi-layered, implantable mesh envelope with an expanding bounding volume that is fillable with soft tissue or soft tissue-like material to the size of the removed tissue or desired tissue replacement during surgery. The 3DME device 100 comprises a three dimensional mesh shaped as an envelope, balloon, disc, or any other 3D bounding volume, including stock sizes, patient-specific or customized to fit the patient. The 3DME device 100 may be manufactured using 3D printing, 3D bioprinting, forming, molding, folding, suturing, crafting, fabricating, gluing, or similar processes. The 3DME device 100 is manufactured from one of or a combination of a biocompatible soft tissue or soft tissue-like material that is derived from either native human or animal tissue sources (e.g. extracellular matrix, dermis, small intestinal submucosa, gastrointestinal submucosa, pericardium, amniotic membrane, tendon, or other bioactive components such as collagen, hyaluronic acid, fibrin, elastin, etc.) or synthetic materials (polyethylene glycol-based hydrogels, polylactic acid and polyglycolic acid scaffolds, polycaprolactone, etc.) or hybrid constructs composed of both natural and synthetic components.

The 3DME device 100 may be absorbable, non-absorbable, or a combination thereof based on clinical needs. Different internal and external components of the 3DME device 100 may degrade at different times. Some layers may be degradable and others may not be. In one embodiment, portions of the 3DME device 100 could be absorbable, non-absorbable, or have varying rates of absorbability. For example, an outer layer could degrade in 30 days, a middle layer in 60 days, and a core in 90 days. This is achieved by using degradable native human or animal derived tissue components or synthetic degradable material components. The filling of a soft tissue cellularized material such as lipoaspirate, adipose-derived stem cells, nanofat, stromal vascular fraction, platelet rich plasma, or other soft tissue cellularized or non-cellularized materials. In one example, the filling material may be alloclae or a similar structural adipose filler made from sterilized, processed donor fat (adipocytes) and extracellular matrix (ECM). The 3DME device 100 may also be filled with a non-cellularized soft tissue injectable and/or filler. The 3DME device 100 may be completely filled or partially filled and is refillable. The 3DME device 100 can be implanted through a minimally invasive surgical procedure (e.g. through a cannula or a funnel) or through an open surgical procedure. The 3DME can be filled prior to implantation, after implantation during the same surgical procedure, or after the surgical procedure, or some combination thereof.

The bounding volume of the 3DME device 100 can be designed to stretch or expand only to a desired volume or shape or can be designed to conform to a specific anatomical shape in the body. This is achieved by using a micro-lattice mesh architecture that is small enough to keep the cellularized infill material inside the bounding volume. Each compartment can be filled at the same time or sequentially over time, including each compartment being filled partially, fully, or more than once. Each port accesses a compartment so that the soft tissue, or soft tissue-like, filler material can be injected into each compartment at the same time, or sequentially (e.g. a specific port for a specific compartment) or some combination thereof. The ports can have access from outside the body, after implantation, via percutaneous injection or transcutaneous port access, to allow for sequential filling over time, or filling multiple times.

The mesh material and soft tissue or soft tissue-like filler material can be fabricated together through 3D printing or 3D bioprinting. The 3DME device 100 can be constructed with variable stiffness, elasticity, and mechanical strength by adjusting the mesh design and internal strut design. The 3DME is designed so that transcutaneous monitoring may be used to assess the vascularity of the filler content to best time additional fills.

Advantages of the 3DME device 100 include the addition of mesh envelope compartments, or layers, with ports accessible outside the body (e.g. after surgery), which allows for sequential filling of adipose, or adipose-like material, over multiple time points (e.g. days, weeks or months in between filling) which allows for engraftment of the adipose, or adipose-like (e.g. lipoaspirate) material into the host tissue supplying adequate oxygen or nutrient delivery. The addition of mesh envelope compartments, or layers, also allows for sequential filling of adipose, or adipose-like material, into specified compartments, layers (e.g. pods, regions, etc.). The 3DME device 100 may be personalized or customized to the patient, anatomic area, or indication.

These advantages allow the 3DME device 100 to feel like soft tissue, regenerate into “self”, and have minimal or no foreign body response from the patient's immune system. The 3DME device 100 allows for a real-time custom shape immediately in the operating room. An approximate size 3DME device 100 is customizable to the size of the removed tissue or desired tissue replacement shape and volume during a minimally invasive surgery.

The 3DME device 100 keeps the cellularized or non-cellularized material (e.g. lipoaspirate) in the desired bounding volume without it leaching out to undesired locations in the body, while allowing for nutrient diffusion (oxygen, glucose, amino acids, fatty acids and lipids, hormones and growth factors, ions and electrolyte, etc.). The 3DME device 100 provides structural support of the soft cellular tissue when needed. This is achieved by using a micro-lattice mesh architecture that is large enough to allow for nutrient diffusion.

In use, the 3DME device 100 may be compressed to fit through a small incision, cannula, funnel, etc., for insertion via a minimally invasive surgery. Once positioned within the body, the plurality of compartments are sequentially filled with adipose or adipose-like material into specific layers or compartments of an implanted mesh envelope through the transcutaneous external ports that are accessible outside the body or via percutaneous injection. Alternatively, one or any of the plurality of compartments may be prefilled or partially prefilled prior to implantation. This would allow pre-manufacture the 3DME device 100 with at least one chamber already filled and it would be shelf stable. In this case, not all chambers would comprise a port.

The ports are tube or tube-like apparatuses that allow for the filling, or injection of an adipose or adipose-like material into the mesh envelope, which is accessible outside the body. The ports may also be constructed within the mesh wall membrane. This allows for predictable shaping and support of soft tissue grafting (e.g. lipoaspirate), as with alloplastic implants currently used, and for tissue regeneration by using a biocompatible 3DME material in combination with a cellularized infill material. The soft tissue or soft-tissue-like material can be injected sequentially over time, at controlled volumes, and into specific locations (e.g. layers or compartments) within the mesh envelope.

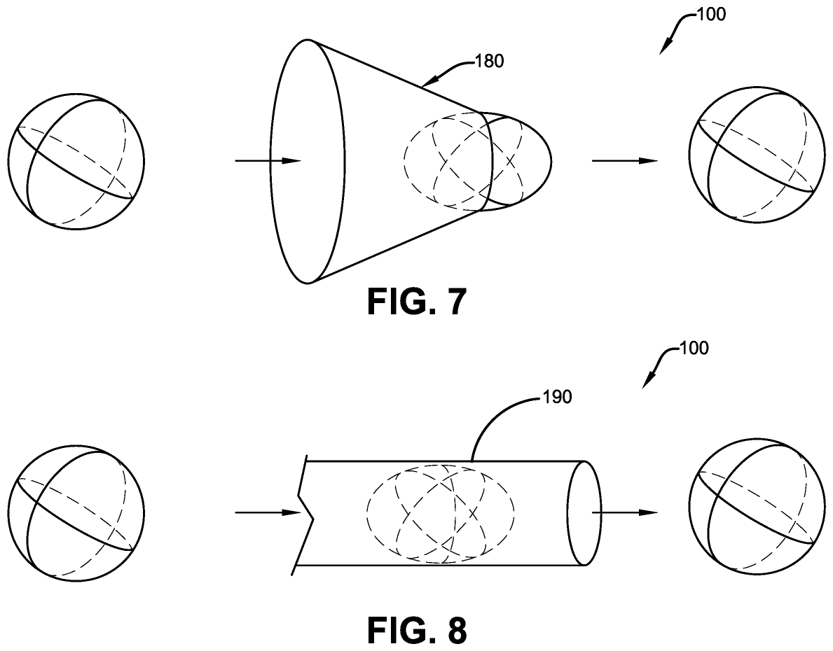

FIGS. 7-8 and 12A-12F illustrate a method of repairing a soft tissue defect. The method begins by constructing a 3DME device 100 as discussed supra. The 3DME device 100 comprises a mesh wall membrane 110, at least one compartment 120 formed within the mesh wall membrane 110 comprising an expandable volume 130, and a port 140 for accessing the expandable volume 130 within the at least one compartment 120. The 3DME device 100 may alternatively comprise a plurality of compartments 120A-D. as illustrated in FIGS. 10-11.

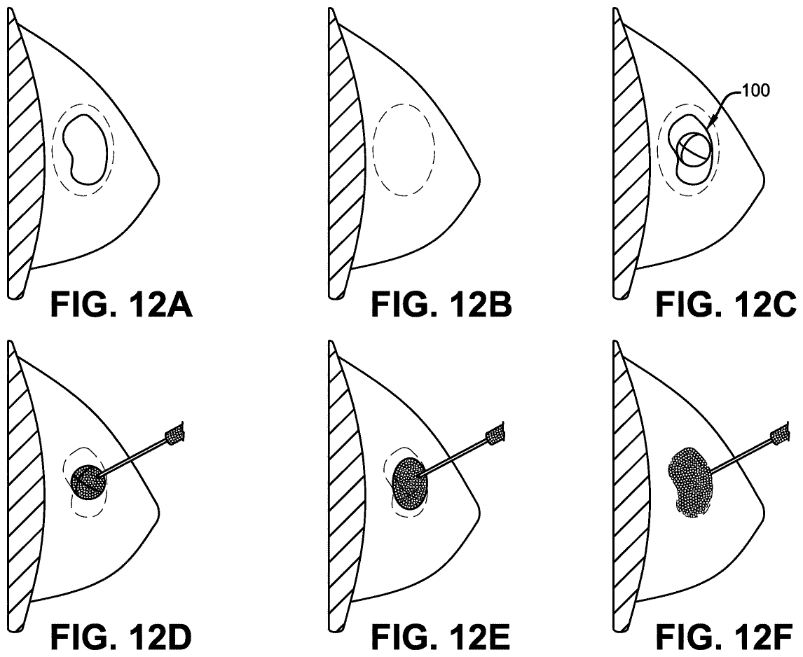

Next, the 3DME device 100 may be implanted into a tissue cavity within a body. The 3DME device 100 may be collapsed to be minimally invasively implanted via a funnel 180 or a cannula 190 as illustrated in FIGS. 7-8. Once positioned within the cavity, a filler material 160 is injected into the at least one compartment 120 or the plurality of compartments 120A-D until the expandable volume completely fills the tissue cavity as illustrated in FIG. 12E and F. As noted previously, one or any of the plurality of compartments may be prefilled or partially prefilled prior to implantation.

Compared to the 2D mesh scaffold products, the 3DME device 100 has 3D shape (i.e. not just a flat sheet), can be customized to fill the three-dimensional defect, and can be filled with a cellularized or non-cellularized soft tissue material. Compared to the 3D printed scaffold products, the 3DME device 100 has 3D shape (i.e. not just a flat sheet), can be customized to fill the three-dimensional defect, and can be filled with a cellularized soft tissue material. Compared to silicone or saline filled silicone implants, the 3DME device 100 can be degradable, combined with cellular or non-cellularized material, and can regenerate into native tissue. Also, the 3DME device 100 eliminates the negative aspects of implanting long-lived devices made of silicone such as the interaction between the body and the polydimethylsiloxane shell of the implant. Compared to breast tissue expanders, the 3DME device 100 can be degradable, combined with cellular or non-cellularized material, and can regenerate into native tissue. Further, the 3DME device 100 can have a radiodensity that is visible under medical imaging. For example, if the 3DME device 100 was implanted in a lumpectomy cavity in a breast, it would be visible under CT scan or X ray.

Compared to soft tissue injectables or fillers, the 3DME device 100 can keep the injected material within a specific bounding volume. The bounding volume can be expandable, customizable, personalized, or of a stock shape or size. The 3DME device 100 can also provide structural support to the injectable material. Unbounded fillers tend not to keep a negative space open. Compared to a mesh envelope, the 3DME device 100 can have a smaller pore size to contain an injectable cellularized or non-cellularized material while still allowing for nutrient transport. The bounding volume can be expandable, customizable, or personalized using the 3DME device 100. Compared to a soft tissue scaffold with cellular injection, the 3DME device 100 will have a bounding volume that can be expandable, customizable, or personalized, that will help hold the injected cellularized material in place while still allowing for nutrient transport. Compared to a soft tissue scaffold without cellular injection, the 3DME device 100 will allow for the injection of a cellularized material, will have a bounding volume that can be expandable, customizable, or personalized, that will help hold the injected cellularized material in place while still allowing for nutrient transport.

It is contemplated that the 3DME device 100 constructed in accordance with the present invention will be tailored and adjusted by those of ordinary skill in the art to accommodate various levels of performance demand imparted during actual use. Accordingly, while this invention has been described by reference to certain specific embodiments and examples, it will be understood that this invention is capable of further modifications. This application is, therefore, intended to cover any variations, uses or adaptations of the invention following the general principles thereof, and including such departures from the present disclosure as come within known or customary practice in the art to which this invention pertains and fall within the limits of the appended claims.

Notwithstanding the foregoing, the 3DME device 100 of the present invention and its various structural components can be of any suitable size, shape, and configuration as is known in the art without affecting the overall concept of the invention, provided that it accomplishes the above stated objectives. One of ordinary skill in the art will appreciate that the 3DME device 100 and its various components and material, as shown in the FIGS. are for illustrative purposes only, and that many other shapes and sizes of the 3DME device 100 are well within the scope of the present disclosure. Although the dimensions of the 3DME device 100 are important design parameters, the 3DME device 100 and its components may be of any shape or size that ensures optimal performance during use and/or that suits user need and/or preference.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A three dimensional mesh envelope device comprising:
a mesh wall membrane;
at least one compartment formed within the mesh wall membrane comprising an expandable volume;
a filler material; and
a port for accessing the expandable volume within the at least one compartment.

2. The three dimensional mesh envelope device of claim 1, wherein the mesh wall membrane is a micro lattice structure.

3. The three dimensional mesh envelope device of claim 1, wherein the mesh wall membrane is a three dimensionally bio-printed mesh.

4. The three dimensional mesh envelope device of claim 1, wherein the mesh wall membrane is configured to permit nutrient diffusion across the mesh wall membrane.

5. The three dimensional mesh envelope device of claim 1, wherein the at least one compartment is configured to retain the filler material within the expandable volume.

6. The three dimensional mesh envelope device of claim 1, wherein the mesh wall membrane is absorbable.

7. The three dimensional mesh envelope device of claim 1, wherein the mesh wall membrane is a biocompatible soft tissue membrane.

8. The three dimensional mesh envelope device of claim 1, wherein the mesh wall membrane is a synthetic or a hybrid membrane.

9. The three dimensional mesh envelope device of claim 1 further comprising a plurality of struts within the expandable volume of the at least one compartment.

10. The three dimensional mesh envelope device of claim 9, wherein the plurality of struts are load bearing struts.

11. A three dimensional mesh envelope device comprising:
a mesh wall membrane;
a plurality of compartments formed within the mesh wall membrane each compartment comprising an expandable volume;
a filler material; and
a port for accessing the expandable volume within each compartment.

12. The three dimensional mesh envelope device of claim 1, wherein the expandable volume of each compartment is different.

13. The three dimensional mesh envelope device of claim 1, wherein the plurality of compartments are oriented in a layered configuration.

14. The three dimensional mesh envelope device of claim 1, wherein each of the plurality of compartments are configured to prevent leaching of the filler material while permitting nutrient diffusion across the mesh wall membrane to nourish the filler material.

15. The three dimensional mesh envelope device of claim 1, wherein each port comprises a tubing for injecting the filler material.

16. The three dimensional mesh envelope device of claim 1, wherein the expandable volume of each of each of the plurality of compartments is adjustable.

17. The three dimensional mesh envelope device of claim 1, wherein the filler material is a harvested adipose or lipoaspirate material.

18. A method of repairing a soft tissue defect comprising the steps of:
constructing three dimensional mesh envelope device comprising a mesh wall membrane and at least one compartment formed within the mesh wall membrane comprising an expandable volume; and
implanting the three dimensional mesh envelope device into a tissue cavity within a body.

19. The method of repairing a soft tissue defect of claim 18 further comprising the step of injecting a filler material into the at least one compartment after implantation via a port.

20. The method of repairing a soft tissue defect of claim 18 further comprising the step of introducing a filler material into the at least one compartment prior to implantation.
